(19) United States
(12) Patent Application Publication
(10) Pub. No.: US 2017/0306322 A1
(43) Pub. Date: Oct. 26, 2017

(54) METHODS AND MATERIALS FOR IDENTIFYING COMPOUNDS PROMOTING TRANSLATIONAL READ-THROUGH OF NONSENSE MUTATIONS

(71) Applicant: Evolva SA, Reinach (CH)

(72) Inventors: Jutta HEIM, Ramlinsburg (CH); Sanne JENSEN, Frederiksberg (DK); Jens KLEIN, Oberwil (CH); Walden Emil BJORN-YASHIMOTO, Copenhagen (DK)

(21) Appl. No.: 15/101,553

(22) PCT Filed: Dec. 4, 2014
(86) PCT No.: PCT/EP2014/076657

§ 371 Date: Jun. 3, 2016

Related U.S. Application Data
(60) Provisional application No. 61/911,764, filed on Dec. 4, 2013.

(51) Int. Cl. C12N 15/10; C12Q 1/68; C12N 15/09

(52) U.S. Cl. CPC C12N 15/1086; C12Q 1/6897; C12Q 1/6827; C12Q 1/6806; C12N 15/09; A61K 2300/00

(57) ABSTRACT

Methods and materials for identification of compounds that allow translation read-through of nonsense mutations in recombinant microorganisms and mammalian cells.

### CROSS-REFERENCE TO RELATED APPLICATIONS

This application claims the benefit of U.S. Provisional Application No. 61/911,764, filed Dec. 4, 2013, the disclosure of which is incorporated by reference in its entirety.

### TECHNICAL FIELD

This invention relates to methods and materials for identification of compounds that allow translation read-through of nonsense mutations.

### BACKGROUND

Nonsense mutations are mutations that result in the introduction of a stop codon, leading to premature termination of the translation of mRNA by the ribosome and production of a truncated protein. Nonsense mutations are the cause of over 1800 distinct genetic disorders, including Duchenne muscular dystrophy, blood diseases such as hemophilia, lysosomal storage disorders, ocular anomalies such as retinal dystrophy, cystic fibrosis, DNA repair disorders, skin disorders and nervous system disorders. Somatic nonsense mutations in tumor suppressor genes also are frequent in cancer. For example, nonsense mutations have been identified in the following tumor suprressor genes: APC (adenomatous polyposis coli) (colon cancer), p53 (multiple cancers including breast, prostate, and lung), RB1 (retinoblastoma), p16 (multiple cancers including pancreatic cancer), and PTEN (multiple cancers including glioblastomas, prostate, and breast cancer).

No cures exist for a majority of disorders caused by nonsense mutations, and instead, treatment regimens are typically symptomatic (e.g., enzyme replacement therapy). However, restoring at least a portion of the target protein production may reduce severity of the disease phenotype. Aminoglycoside antibiotics such as gentamycin have been identified as compounds that allow suppression of premature stop codons, such that translation continues through the stop codon (read-through). Another compound, PTC124 (PTC Therapeutics) is currently in clinical trials for treatment of cystic fibrosis, muscular dystrophy, and hemophilia. There is a need for further compounds that can allow production of functional proteins in patients with genetic disorders due to a nonsense mutation.

### SUMMARY

This document is based on the discovery of methods of identifying compounds that lead to translational read-through of a nonsense mutation and thus restore gene function.

In an aspect, this document relates to methods for identifying compounds that permit translational read-through of nonsense mutations, comprising: (a) providing a recombinant microorganism, the microorganism comprising at least one nucleic acid construct, the construct comprising a regulatory region operably linked to a nucleic acid sequence containing a nonsense mutation, wherein the nucleic acid sequence containing the nonsense mutation is operably linked to a nucleic acid sequence encoding a first reporter gene; (b) contacting the recombinant microorganism with a test compound; and (c) determining if the microorganism produces the reporter.

In another aspect, the recombinant microorganism comprises a second nucleic acid construct comprising a promoter operably linked to a native gene containing a native stop codon that is operably linked to a second reporter gene, wherein the second reporter gene is a different reporter gene than the first reporter gene.

In yet another aspect, this document relates to pharmaceutical compositions comprising one or more of the compounds identified by the methods described herein, and uses of the compositions for treating a subject with a genetic disorder caused by nonsense mutation. In an embodiment, the compositions and compounds permit translational read-through of nonsense mutations.

In yet another aspect, this document relates to nucleic acid constructs comprising a regulatory region operably linked to a nucleic acid sequence containing a nonsense mutation, wherein the nucleic acid sequence containing the nonsense mutation is operably linked to a nucleic acid sequence encoding a first reporter gene. In an embodiment, a nucleic acid construct comprises a regulatory region operably linked to a native gene containing a native stop codon that is operably linked to a second reporter gene, wherein the second reporter gene is a different reporter gene than the first reporter gene.

In yet another aspect, this document relates to recombinant microorganisms comprising at least one nucleic acid construct, the construct comprising a regulatory region operably linked to a nucleic acid sequence containing a nonsense mutation, wherein the nucleic acid sequence containing the nonsense mutation is operably linked to a nucleic acid sequence encoding a first reporter gene.

In yet another aspect, this document relates methods of treating a condition caused by a nonsense mutation in a subject, comprising administering to the subject a pharmaceutically effective amount of the compositions described herein.

### DESCRIPTION OF DRAWINGS

FIG. 1 shows a schematic of an assay for detecting compounds that allow translational read through of stop codons.

FIG. 2 shows a schematic of reporter constructs with pSCR plasmid identity numbers and the nucleotide sequence of the APC fragment.

FIG. 3 shows results of the assay using an APC gene fragment containing a nonsense mutation. FIG. 3A demonstrates that the presence of the stop codon prevents transcription of the marker gene. FIG. 3B demonstrates that the presence of G418 (Geneticin®) allows translation read-through of the APC gene. FIG. 3C is a bar graph indicating that paromomycin treatment leads to suppression of the APC stop codon and production of URA3 protein.

FIG. 4 shows the results of the Dual Luciferase® Assay experiment demonstrating that G418 and paromomycin are both able to induce luciferase activity and validates the APC stop codon luciferase reporter system (samples were tested in triplicate and data was analyzed by GraphPad® with error bars showing mean with 95% confidence interval).

FIG. 5 shows representative schematics of luciferase constructs for use in mammalian cell based systems to detect translation read-through of stop codons. FIG. 5A is a schematic of a mammalian cell based dual luciferase construct for detection of compounds that allow translational read-through of stop codons. FIG. 5B shows the APC reporter construct expressing from a strong constitutive CMV promoter and harboring the ZeoR selection gene. FIG. 5C is a schematic of plasmid pSCR-220 with a Renilla luciferase gene under PCMV control and the puroR gene for selection.

FIG. 6 shows an example of a transfection protocol of HCT-116 cells using GeneJuice® reagent.

FIG. 7 shows number of CEYs display growth in media lacking uracil, consistent with URA3 reporter transcription (data tested in triplicate and analyzed by GraphPad Prism® software. Growth in SCgal-HLU and SCgal-HLUM is indicated as A600, % of control strain (EFSC2944).

FIG. 8 shows the structure and characterization of yeast derived stop codon read-through compound CEY4906 132 211.

FIG. 9 shows the structure and characterization of yeast derived stop codon read-through compound CEY4906 147 239 (GC-0054).

FIG. 10 shows the structure and characterization of yeast derived stop codon read-through compound CEY4905 77-78 208.

FIG. 11 shows the structure and characterization of yeast derived stop codon read-through compound CEY4905 13-15 248.

FIG. 12 shows the structure and characterization of yeast derived stop codon read-through compound CEY4905 13-15 266 RT 5.5.

FIG. 13 shows the structure and characterization of yeast derived stop codon read-through compound CEY4906 66-67 266.

FIG. 14 shows the structure and characterization of yeast derived stop codon read-through compound CEY4906 45-46 238.

FIG. 15 shows the structure and characterization of yeast derived stop codon read-through compound CEY4906 71-75 445.

FIG. 16 shows examples of total ion chromatograms of transformed and non-transformed yeast. Total ion chromatogram (positive ion mode) (TIC ESI+) of a crude extract of non-transformed, control yeast (FIG. 16A) (CEY4923) and of transformed yeast containing artificial chromosomes (FIG. 16B) (CEY4905).

FIG. 17 shows total ion chromatogram (positive ion mode) (TIC ESI+) of a fraction (74) of a crude extract of non-transformed, control yeast (FIG. 17C), CEY4923, and of transformed yeast containing artificial chromosomes (FIG. 17B), CEY4906. TIC of the purified YAC dependent compound (CEY4906_71-75_445) purified from the active fraction 74 of CEY4906 (FIG. 17A).

Like reference symbols in the various drawings indicate like elements.

### DETAILED DESCRIPTION

Unless otherwise defined, all technical and scientific terms used herein have the same meaning as commonly understood by one of ordinary skill in the art to which the invention belongs. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and are not intended to be limiting. Other features and advantages of the invention will be apparent from the following detailed description. Methods well known to those skilled in the art can be used to construct genetic expression constructs and recombinant cells according to this invention. These methods include in vitro recombinant DNA techniques, synthetic techniques, in vivo recombination techniques, and polymerase chain reaction (PCR) techniques. See, for example, techniques as described in Maniatis et al., 1989, MOLECULAR CLONING: A LABORATORY MANUAL, Cold Spring Harbor Laboratory, New York; Ausubel et al., 1989, CURRENT PROTOCOLS IN MOLECULAR BIOLOGY, Greene Publishing Associates and Wiley Interscience, New York, and PCR Protocols: A Guide to Methods and Applications (Innis et al., 1990, Academic Press, San Diego, Calif.).

Before describing the present invention in detail, a number of terms will be defined. As used herein, the singular forms “a”, “an”, and “the” include plural referents unless the context clearly dictates otherwise. For example, reference to a “nucleic acid” means one or more nucleic acids.

It is noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

As used herein, the terms “polynucleotide”, “nucleotide”, “oligonucleotide”, and “nucleic acid” can be used interchangeably to refer to nucleic acid comprising DNA, RNA, derivatives thereof, or combinations thereof.

This document describes materials and methods that can be used to identify compounds that lead to translational read-through of a nonsense mutation and thus restore gene function.

In an aspect, this document relates to methods for identifying compounds that permit translational read-through of nonsense mutations, the method comprising: (a) providing a recombinant microorganism, the microorganism comprising at least one nucleic acid construct, the construct comprising a regulatory region operably linked to a nucleic acid sequence containing a nonsense mutation, wherein the nucleic acid sequence containing the nonsense mutation is operably linked to a nucleic acid sequence encoding a first reporter gene; (b) contacting the recombinant microorganism with a test compound; and (c) determining if the microorganism produces the reporter.

In another aspect, the recombinant microorganism comprises a second nucleic acid construct comprising a promoter operably linked to a native gene containing a native stop codon that is operably linked to a second reporter gene, wherein the second reporter gene is a different reporter gene than the first reporter gene.

In yet another aspect, this document relates to nucleic acid constructs comprising a regulatory region operably linked to a nucleic acid sequence containing a nonsense mutation, wherein the nucleic acid sequence containing the nonsense mutation is operably linked to a nucleic acid sequence encoding a first reporter gene. In an embodiment, a nucleic acid construct comprises a regulatory region operably linked to a native gene containing a native stop codon that is operably linked to a second reporter gene, wherein the second reporter gene is a different reporter gene than the first reporter gene.

The term “reporter gene” refers to a gene or nucleic acid sequence attached to a regulatory region of another gene that is introduced into a recipient host, and when expressed, can be any detectable or measurable protein. In some embodiments, the reporter can be detected non-invasively (i.e., a fluorescent or luminescent marker). Non-limiting examples of reporters can be green fluorescent protein (GFP), HcRed, DsRed, mCherry, cyan fluorescent protein (CFP), yellow fluorescent protein (YFP), blue fluorescent protein (BFP), glutathione-S-transferase (GST), horseradish peroxidase (HRP), chloramphenicol acetyltransferase (CAT) beta-galactosidase, beta-glucuronidase (GUS), firefly luciferase, renilla luciferase, selectable auxotrophic marker proteins including orotidine-5′ phosphate decarboxylase (encoded by yeast URA3), phosphoribosylaminoimidazole carboxylase (encoded by yeast ADE2) and selectable antibiotic resistance marker proteins including the Streptoalloteichus hindustanus ble gene product conferring resistance to Zeocin™, or reporter genes that confer resistance to antibiotics, such as ampicillin, neomycin, puromycin, methotrexate, or tetracyclin.

The terms “regulatory region,” “promoter” or “promoter element” as used herein, refer to a DNA sequence which when ligated to a nucleotide sequence of interest is capable of controlling the transcription of the nucleotide sequence of interest into mRNA. A regulatory region is typically, though not necessarily, located 5′ (i.e., upstream or infront) of a nucleotide sequence of interest whose transcription into mRNA it controls, and provides a site for specific binding by RNA polymerase and other transcription factors for initiation of transcription.

Promoters may be constitutive or regulatable. The term “constitutive” when made in reference to a promoter means that the promoter is capable of directing transcription of an operably linked nucleic acid sequence in the absence of a stimulus (e.g., heat shock, chemicals, etc.). In contrast, a “regulatable” or “inducible” promoter is one which is capable of directing a level of transcription of an operably linked nucleic acid sequence in the presence of a stimulus (e.g., heat shock, light, chemicals, etc.) which is different from the level of transcription of the operably linked nucleic acid sequence in the absence of the stimulus.

In some embodiments, the constitutive promoter can be selected from cytomegalovirus promoter, tRNA promoter, 5S rRNA promoters, histone gene promoters, RSV promoter, retrovirus LTR promoter, SV40 promoter, PEPCK promoter, MT promoter, SR-alpha promoter, P450 family promoters, p16 promoter, p21 promoter, PPARG promoter, SCARB1 promoter, AVTA2 promoter, PTN promoter, GALT promoter, T7 promoter, T3 promoter, SP6 promoter, K11 promoter, HIV promoter, yeast ADH1 promoter, yeast PGK1 promoter, yeast TDH3 promoter, yeast TPI1 promoter, and yeast TEF1 promoter.

In other embodiments, the regulatable or inducible promoter can be selected from a galactose inducible promoter, a maltose inducible promoter, a heat shock promoter, a tetracycline inducible promoter, Isopropyl β-D-1-thiogalactopyranoside (IPTG) inducible promoter, copper inducible promoter, methionine inducible promoter, serine inducible promoter.

The term “test compound” as used herein, refers to a compound or any molecule or agent, e.g. protein, non-protein organic compound or pharmaceutical, with the capability of being affected by or affecting the translational read-through of a nonsense mutation and thus restore gene function. There are no particular restrictions as to the test compounds that can be assayed. Such compounds can include, for example, polypeptides, peptides, antibodies, peptidomimetics, peptoids, small inorganic molecules, small non-nucleic acid organic molecules, nucleic acids (e.g., anti-sense nucleic acids, siRNA, oligonucleotides, synthetic oligonucleotides), carbohydrates, or other agents that bind to the target proteins, have a stimulatory or inhibitory effect on, for example, expression of a target gene or activity of a target protein. Compounds thus identified can be used to positively affect the translational read-through of a nonsense mutation and thus restore gene function.

Conventionally, compounds identified that can affect the translational read-through of a nonsense mutation are called a “hit”. The hit can then be used as a scaffold to create variants of the hit, and further evaluate the property and activity of those variant compounds. One skilled in the art will appreciate the utility of using the methods described herein to optimize compound selection by identifying potential hits and identifying or selecting those compounds with the ability to lead to the translational read-through of a nonsense mutation and thus restore gene function.

Test compounds can be selected or screened from, for example, libraries of small molecules or expression libraries; biological libraries; peptoid libraries; spatially addressable parallel solid phase or solution phase libraries; synthetic libraries. The methods described herein include methods (also referred to as “screening assays”) for identifying compounds that lead to the translational read-through of a nonsense mutation and thus restore gene function. In general, screening assays involve assaying the effect of a test compound on expression or activity of a target nucleic acid or target protein (i.e., the reporter gene) in a test sample. Expression or activity of the reporter gene in the presence of the test compound or agent can be compared to expression or activity of the reporter gene in a control sample (i.e., a sample containing the reporter gene that is incubated under the same conditions, but without the test compound). A change in the expression or activity of the target nucleic acid or target protein (i.e. reporter gene) in the test sample compared to the control indicates that the test compound or test agent modulates expression or activity of the reporter gene and is able to affect the translational read-through of a nonsense mutation and thus a candidate compound or agent (i.e. hit or lead).

Test compounds can be assayed in a highly parallel fashion using multiwell plates by placing the compounds either individually in wells or testing them in mixtures, and the reporter can then be measured or detected with a plate reader.

The term “determining” as used herein, refers to quantitatively or qualitatively detecting or measuring the effect of a test compound on the translational read-through of a nonsense mutation.

In yet another aspect, this document relates methods of treating a condition caused by a nonsense mutation in a subject, comprising administering to the subject a pharmaceutically effective amount of the compositions described herein.

The term “nonsense mutation” as used herein, refers to a single base pair substitution that prematurely codes for a stop in amino acid translation (i.e., a stop codon). A nonsense mutation is the substitution of a single base pair that leads to the appearance of a stop codon where previously there was a codon specifying an amino acid. The presence of this premature stop codon results in the production of a truncated, incomplete, and usually nonfunctional protein product. Nonsense mutations can cause genetic disease by damaging a gene responsible for a specific protein. Translational read-through of a nonsense mutation allows production of the full-length protein, thereby recovering the normal form of the protein and suppressing the nonsense mutation. Thus, a compound that permits translation read-through could restore gene function and reverse the negative outcome of the genetic disease. Nonsense mutations in a gene are known to cause genetic diseases, for example, dystrophin in Duchenne muscular dystrophy (also see Table 1). Additional genetic diseases attributable to nonsense mutations include, but are not limited to, blood diseases such as hemophilia, lysosomal storage disorders, ocular anomalies such as retinal dystrophy, cystic fibrosis, DNA repair disorders, skin disorders and nervous system disorders. Somatic nonsense mutations in tumor suppressor genes also are frequent in cancer. For example, nonsense mutations have been identified in the following tumor suprressor genes: APC (adenomatous polyposis coli) (colon cancer), p53 (multiple cancers including breast, prostate, and lung), RB1 (retinoblastoma), p16 (multiple cancers including pancreatic cancer), and PTEN (multiple cancers including glioblastomas, prostate, and breast cancer). In cystic fibrosis the chloride ion channel CFTR nonsense mutations G542X or W1282X have been identified that prevent the synthesis of fully functional protein. In colorectal cancer, about 85% a cases have sporadic mutations and the remainder is germ-line mutations. A key gene mutated in colorectal cancer is adenomatous polyposis coli (APC), a tumor suppressor gene. In around 80% of sporadic and hereditary colorectal cancers APC is non-functional. Over 700 mutations have been identified so far in the APC gene of which about 30% are nonsense mutations inserting a premature stop codon. In somatic mutations hot spots in the APC gene occur at positions 1309 and 1450.

TABLE 1
Examples of diseases related to nonsense/premature stop codon mutations.

| Disease | Gene(s) Affected |
|---|---|
| Ocular anamolies | |
| Autosomal dominant congenital cataract | Major subunit of beta-crystallin; CRYBB1 |
| Early onset retinal dystrophy | Orthodenticle protein homolog; OTX2 |
| Usher syndrome | Myosin VIIa; MYO7A |
| Blood diseases | |
| Beta-thalassemia | Hemogloin; HBB |
| Hemophilia (A and B) | Factor VII; FVII; and Factor IX; FIX |
| Cancer and DNA repair diseases | |
| Colorectal cancer | Adenomatous polyposiscoli; APC |
| Xeroderma pigmentosa | Several genes, among them XPA, B, B |
| Ion channel function diseases | |
| Cystic fibrosis | Cystic fibrosis transmembrane regulator; |
| | CFTR |
| Metabolic disorders | |
| Lysosomal storage disease (>50 disorders) | e.g., mucopolysaccharidosis type I; MPSI |
| Methylmalonic aciduria (MMA) | Methylmalonyl-CoA mutase; MUT |
| Nervous system disorders | |
| Menkes syndrome | Copper-transporting ATPase 1; ATP7A |
| Connective and muscle tissue disorders | |
| Ostegenesis imperfecta (OI) | Type 1 collagen; COL1A1 |
| Duchenne muscular dystrophy | Dystrophin; DMD |
| Cilia disorders | |
| Primary ciliary dyskinesia (PCD) | Thioredoxin; TXNDC3 |
| Skin Diseases | |
| Xeroderma pigmentosum | xeroderma pigmentosum, complementation |
| | group A; XPA, XPAC or XP1 |
| Ichthyosis vulgaris | filaggrin; FLG or ATOD2 |
| Epidermolysis bullosa | keratin 5; KRT5, K5, CK5, DDD, DDD1, EBS2 |
| | or KRT5A |
| RDEB (recessive dystrophic E. bullosa) | collagen VII; COL7A1, EBD1, EBR1 or |
| | EBDCT |
| Erythrokeratoderma | loricrin; LOR |
| Pachyonychia congenita | keratin 6, 16 and 17 |
| Neurofibromatosis | neurofibromin; NF1, WSS, NFNS or VRNF |

In yet another aspect, this document relates to recombinant microorganisms comprising at least one nucleic acid construct, the construct comprising a regulatory region operably linked to a nucleic acid sequence containing a nonsense mutation, wherein the nucleic acid sequence containing the nonsense mutation is operably linked to a nucleic acid sequence encoding a first reporter gene.

A number of prokaryotes and eukaryotes are suitable for use in constructing the recombinant microorganisms or recombinant hosts described herein, e.g., gram-negative bacteria, yeast and fungi, and mammalian cell lines. Exemplary prokaryotic and eukaryotic species are described in more detail below. However, it will be appreciated that other species may be suitable. For example, suitable species may be in Saccharomycetes. Additional suitable species may be in a genus selected from the group consisting of Agaricus, Aspergillus, Bacillus, Candida, Corynebacterium, Escherichia, Fusarium/Gibberella, Kluyveromyces, Laetiporus, Lentinus, Phaffia, Phanerochaete, Pichia, Physcomitrella, Rhodoturula, Saccharomyces, Schizosaccharomyces, Sphaceloma, Xanthophyllomyces and Yarrowia.

Exemplary species from such genera include Lentinus tigrinus, Laetiporus sulphureus, Phanerochaete chrysosporium, Pichia pastoris, Physcomitrella patens, Rhodoturula glutinis 32, Rhodoturula mucilaginosa, Phaffia rhodozyma UBV-AX, Xanthophyllomyces dendrorhous, Fusarium fujikuroi/Gibberella fujikuroi, Candida utilis and Yarrowia lipolytica. In some embodiments, a microorganism can be an Ascomycete such as Gibberella fujikuroi, Kluyveromyces lactis, Schizosaccharomyces pombe, Aspergillus niger, or Saccharomyces cerevisiae. In some embodiments, a microorganism can be a prokaryote such as Escherichia coli, Rhodobacter sphaeroides, or Rhodobacter capsulatus. It will be appreciated that certain microorganisms or hosts can be used to screen and test for compounds of interest in a high throughput manner. In an embodiment, the recombinant microorganism or host is a yeast cell from Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Hansenula polymorpha, Candida boidinii, Arxula adeninivorans, Xanthophyllomyces dendrorhous or Candida albicans species. In another embodiment, the recombinant host is a mammalian cell line such as NIH-313, HEK-293, CHO, COS-7, MDCK, HeLa or HCT-116.

### Pharmaceutical Compositions

In another aspect, this document relates to pharmaceutical compositions comprising one or more of the compounds identified by the methods described herein, and uses of the compositions for treating a subject with a genetic disorder caused by nonsense mutation. In an embodiment, the compositions and compounds permit translational read-through of nonsense mutations.

A test compound that has been screened by a method described herein and determined to positively affect the translational read-through of a nonsense mutation, can be considered a hit or candidate compound (i.e. lead compound or lead). A hit or lead compound that has been screened, and determined to have a desirable effect on the genetic disorder, can be considered a candidate therapeutic agent. Candidate therapeutic agents, once screened in a clinical setting, are therapeutic agents. Candidate therapeutic agents and therapeutic agents can be optionally optimized and/or derivatized, and formulated with physiologically acceptable excipients to form pharmaceutical compositions.

The compounds determined to positively affect the translational read-through of a nonsense mutation can be incorporated into pharmaceutical compositions. Such compositions typically include the compound and a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. A parenteral preparation can be enclosed in ampoules, disposable syringes or multiple dose vials made of glass or plastic.

The invention will be further described in the following examples, which do not limit the scope of the invention described in the claims.

### EXAMPLES

### Example 1. Detecting Compounds that Allow Translational Read-Through of Stop Codons

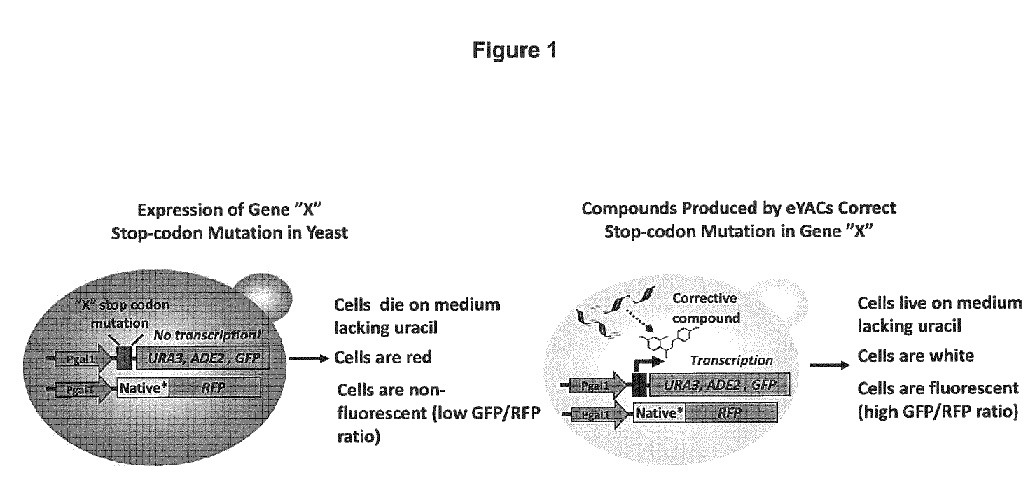

A nucleic acid sequence containing a nonsense mutation was introduced into a yeast expression construct under the transcriptional control of a promoter e.g., an inducible promoter such as a galactose inducible promoter. The nucleic acid sequence containing the nonsense mutation was fused to a first marker gene such as URA3 and/or ADE2 (or other gene that results in an auxotroph if the gene is not transcribed and translated). The plasmid was integrated into an intergenic region in the genome of a modified yeast strain harboring deletion of ura3 and/or ade2 using conventional LiAc yeast transformation methods. Integrants were selected on media plates containing the antibiotic Hygromycin B or lacking histidine selecting for the marker present on the expression plasmid. Integrated strains were incubated with test compounds and plated on relevant growth media. Suitable test compounds include, for example, libraries of small molecules or expression libraries. If the premature stop codon was overridden in the presence of the test compound, then the ura3 and/or ade2 gene was transcribed and resulted in production of URA3 and/or ADE2 protein, respectively. Production of URA3 allowed the yeast cells to grow and form colonies on media lacking uracil after 2-4 days incubation at 30° C. Production of ADE2 conferred a white coloration of the yeast colonies and allowed the yeast cells to grow and form colonies on media plates lacking adenine after 2-4 days incubation at 30° C. (see FIG. 1)

In some embodiments, a two-reporter system is used wherein a second construct is a control to make sure the test compound does not alter translation of native stop codons. In such a system, a first nucleic acid contains a promoter operably linked to a stop codon mutation to be tested that is operably linked to a nucleic acid encoding a first reporter gene (e.g., a firefly luciferase (Fluc) or green fluorescent protein (GFP)). The yeast also includes a second nucleic acid construct that includes a promoter operably linked to a native gene containing a native stop codon that is operably linked to a nucleic acid encoding a second reporter gene that is different from the first reporter (e.g., a renilla luciferase (Rluc) or red fluorescent protein (RFP)). See FIG. 1. In the two-reporter system, a low ratio of the first reporter gene signal compared to the second reporter gene signal (i.e., low Fluc/Rluc ratio or low GFP/RFP ratio) would indicate a compound that does not result in translation read-through of the test stop codon. A high ratio of the first reporter gene signal compared to the second reporter gene signal (i.e., high Fluc/Rluc ratio or high GFP/RFP ratio) would indicate a compound that results in translation read-through of the test stop codon, but not the native stop codon, and therefore would be a positive hit. Using such a second construct is a control to make sure a test compound does not alter translation of native stop codons.

### Example 2. Functional Yeast-Based Stop Codon Suppression Assay—APC Target

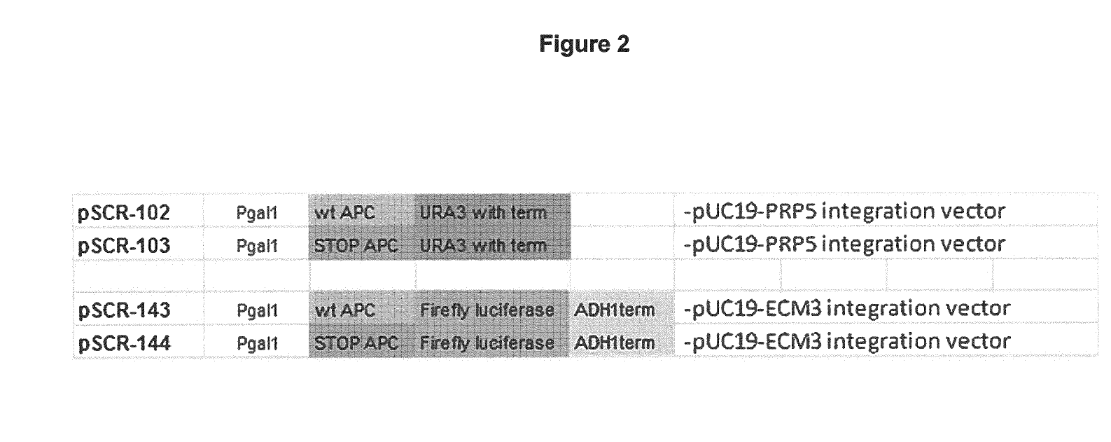

The method was tested using the APC and p53 genes containing nonsense mutations. In the case of APC, a gene fragment with a premature stop codon mutation at amino acid C1450 (wild-type APC is the control and C1450X, which codes for a stop codon, was tested), which has been identified in colon cancers, was introduced into a yeast expression vector under the control of the galactose-inducible GAL1 promoter. The wildtype APC fragment was included as control. Strain EFSC2686 (mat a) and strain EFSC2683 (mat alpha) contain a functional premature stop codon assay based on the premature stop codon C1450X in the adenomatous polyposis coli (APC) gene. The two strains harbor two integrated reporter constructs (URA3 and yeast codon-optimized Firefly luciferase, FLuc), where a fragment carrying the C1450X mutation with 6 APC amino acid residues on each side is inserted in front of and in frame with the reporter genes and after the PGAL1 promoter. See FIG. 2 for a schematic of reporter constructs with pSCR plasmid identity numbers and the sequence of the APC fragment. Plasmids and sequences are shown that contain wild type (wt) APC fragment, used as control, and C1450X (STOP) APC fragment.

WT-APC (SEQ ID NO. 01; bold CGA is codon 1450):
5′-CTAGC ATG CAA ACA GCT CAA ACC AAG CGA GAA GTA
CCT AAA AAT AAA C
STOP-APC (SEQ ID NO. 02; bold TGA is codon C1450X
mutation):
5′-CTAGC AAA ATG CAA ACA GCT CAA ACC AAG TGA GAA
GTA CCT AAA AAT AAA C

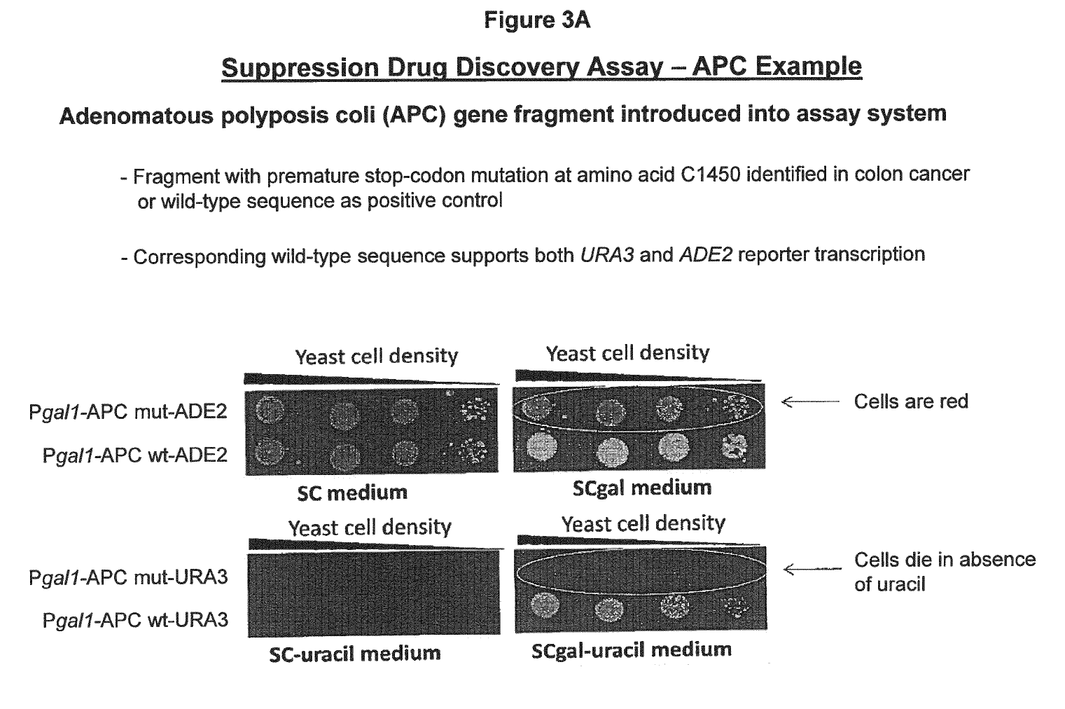

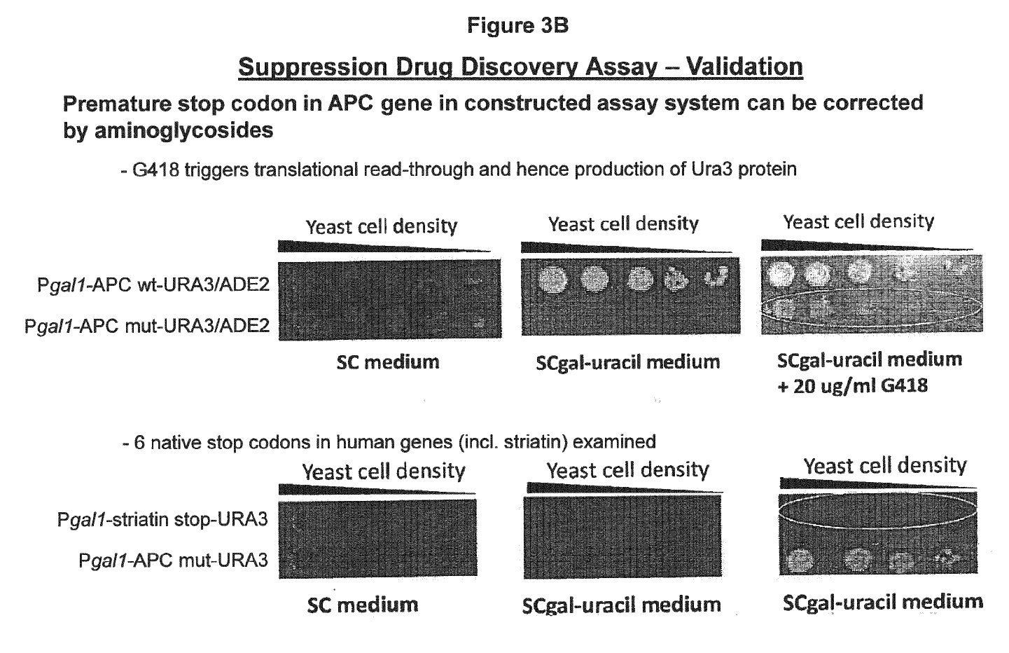

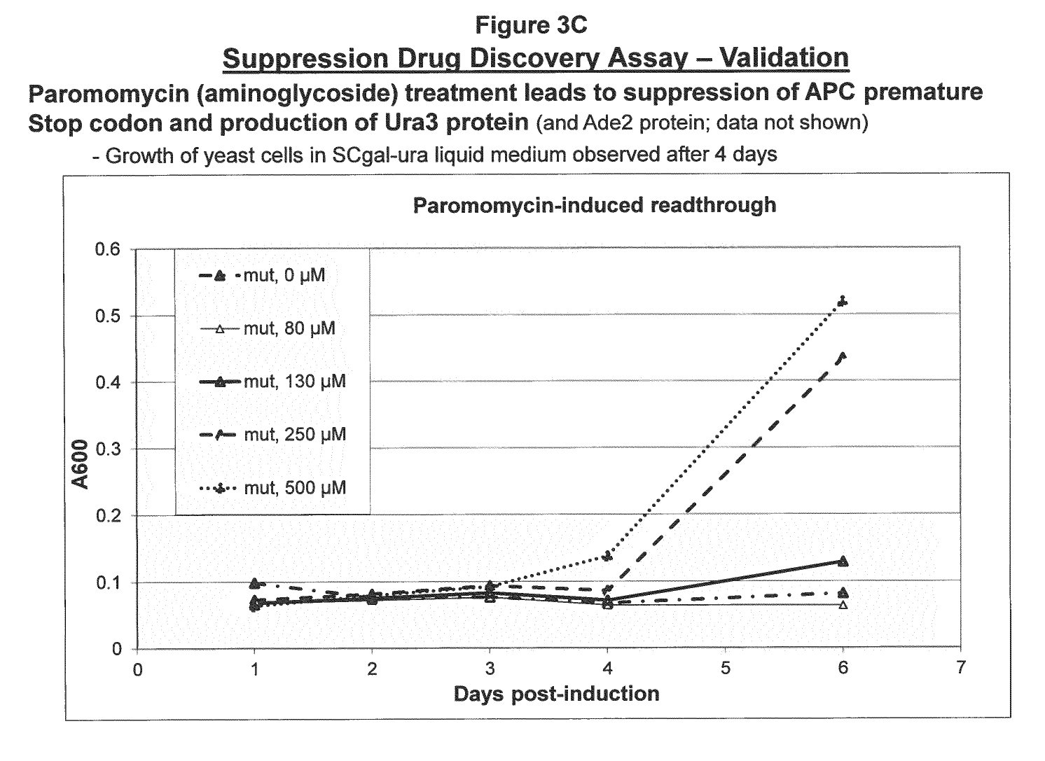

Respective plasmids were integrated into a ade2D/ura3D yeast strain and integrant colonies subjected to spot-test analysis in serial dilution to test survival on media lacking uracil and for color phenotype on synthetic complete (SC) media containing low amounts of adenine. As shown in FIG. 3A, when the APC fragment encoding a stop codon was expressed (SCgal media) cells had a red appearance and failed to support growth on media lacking uracil (SCgal.uracil). This was in contrast to expression of a wildtype APC fragment, which resulted in white colored cells and survival on media lacking uracil. The haploid strains carrying integrated versions of the APC WT fragment grew robustly on SCgal-ura plates and showed white color phenotype on SCgal media. Importantly, strains with mutant APC fragment failed to grow on SCgal-ura media and showed the red phenotype characteristic of an ade2Δ strain. This showed that the assay is “tight” with no detectable background transcription of the URA3 and ADE2 reporter genes in the absence of suppressor.

Next, the yeast strains were tested for read-through of the stop codon in the presence of the aminoglycoside G418. As shown in the spot test analysis of FIG. 3B, the presence of G418 (20 ug/ml) triggered read-through of the stop codon to produce sufficient Ura3p to allow growth on SCgal-ura plates. This showed that the premature stop codon in the APC fragment in the constructed assay above can be corrected by treatment with aminoglycosides (see FIG. 3B). In this case, strains Pgal1-APCwt-URA3/ADE2 and Pgal1-APCmut-URA3/ADE2 were tested by spot-test analysis on galactose-containing media containing 20 ug/ml G418. Following 3-5 days incubation at 30° C., the Pgal1-APCmut-URA3/ADE2 strain formed visible colonies on SCgal-uracil medium.

In a similar set-up, read-through of 6 native stop codons in human genes (V-ATPase, Mdm2, ArhGAP1, DAT and PKCb, and striatin) was examined in front of the URA3 reporter system. In all of the tested cases, no detectable read-through of stop codon was observed i.e., no growth of colonies on SCgal-uracil+20 ug/ml G418. A representative spot-test analysis is shown in FIG. 3B, where a strain containing the native stop codon of the human striatin gene was compared with the strain containing the APC premature stop codon mutant. As evident, there was no detectable suppression of the native stop codon and the strain failed to grow on media lacking uracil supplemented with G418. In contrast, the APC mutant strain showed translational read-through in the presence of G418. The native stop codons in V-ATPase, Mdm2, ArhGAP1, DAT and PKCb were analyzed in a similar fashion and with similar results to striatin. Thus, suppression by G418 appears to be selective for premature stop codons in that there is no detectable suppression observed in the 6 native stop codons tested.

Liquid suppression tests were also conducted with a different aminoglycoside, paromomycin. Read-through of the premature stop codon in APC was also observed after liquid cultivation of the strain in the presence of increasing concentrations of the aminoglycoside paromomycin. Here, strain EFSC2686 was grown in SCgal-ura media in the presence of increasing amounts of drug (0-500 uM) and growth rate was monitored on a daily basis. The presence of ≧250 uM paromomycin supports growth in SCgal media lacking uracil after 4 days incubation (see FIG. 3C). Again, there was no significant background as the strain failed to grow in the set-up in the absence of drug. Similar results were obtained in medium lacking adenine.

These results validate the APC premature stop codon assay (URA3 reporter) under two distinct experimental conditions (liquid and plate-based) using two different aminoglycosides. Importantly, the URA3 reporter has been validated both haploid strains EFSC2683 and EFSC2686 and in the resultant diploid (EFSC2740).

To assess functionality of the Fluc reporter system, a luciferase activity protocol for the Dual Luciferase® Assay kit (Promega) was optimized to be able to test yeast strains in 96-well format. A representative experiment is outlined here. The yeast were incubated for 30° C. overnight. The luciferase assay was performed according to manufacturer's recommended protocol. Briefly, yeast cells were resuspended in SCgal culture media and measured at A600. A 10 uL sample was transferred to a white-opaque-bottom 96-well plate, mixed with 50 uL of passive lysis buffer and incubated at room-temperature for 20-30 minutes with gentle shaking. Next, 50 uL of luciferase assay reagent II and 50 uL of Stop & Glo® reagent were added. The plate was read in a plate reader using a dual luciferase program. The remaining yeast culture was maintained at 30° C. and the luciferase assays was performed daily.

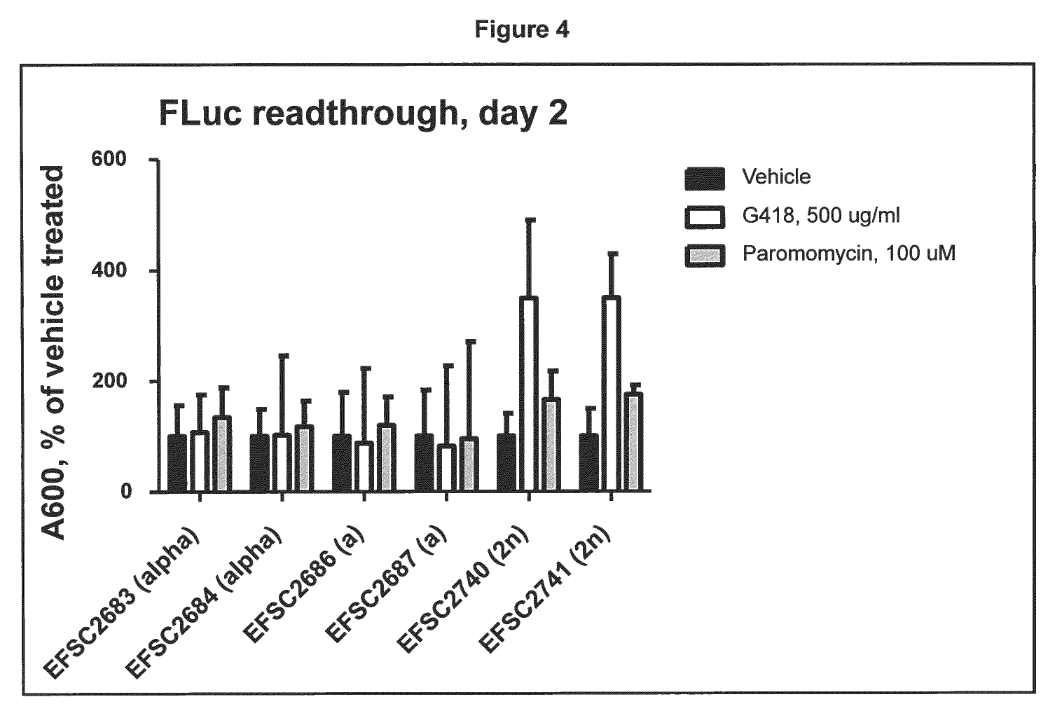

The results of the above Dual Luciferase® Assay experiment are shown in FIG. 4 (samples were tested in triplicate and data was analyzed by GraphPad® with error bars showing mean with 95% confidence interval). Here, 3 different integration sites for the Fluc reporter were analyzed both in a haploid assay strain and in a diploid setting in the presence of aminoglycosides G418 and paromomycin. Diploid strain EFSC2740 showed robust and significant Firefly luciferase activity in the presence of G418 compared to background level with vehicle only (DMSO). Also paromomycin was able to induce luciferase activity. Thus, the APC premature stop codon luciferase reporter system is functional and has been validated with two types of aminoglycosides.

To summarize, the yeast APC stop-codon assay with URA3 and Fluc reporter read-out was functional and validated for use as a primary screening system to identify compounds with translational readthrough activities.

In addition to the APC stop-codon assay, other oncology targets including p53 were analyzed. Here, yeast strains were engineered with P_{GAL1}-P53 STOP (TAA198)-URA3 (EFSC2231 and EFSC2233), P_{GAL1}-P53 STOP (TAG221)-URA3 (EFSC2186 and EFSC2200) and P_{GAL1}-P53 STOP (TGA196)-URA3 (EFSC2232 and EFSC2201) reporter integrated at the PRP5 locus. The strains were tested for suppression with G418 on SCgal-ura plates supplemented with increasing G418 concentrations. In all cases, G418 was able to trigger expression of Ura3p and thus growth on SCgal-ura3 plates; however there appears to be a gradient in the response with TGA showing the best suppression and the following order: TGA>TAG>TAA.

### Example 3. Secondary Mammalian Cell-Based Stop Codon Suppression Assay—APC Target

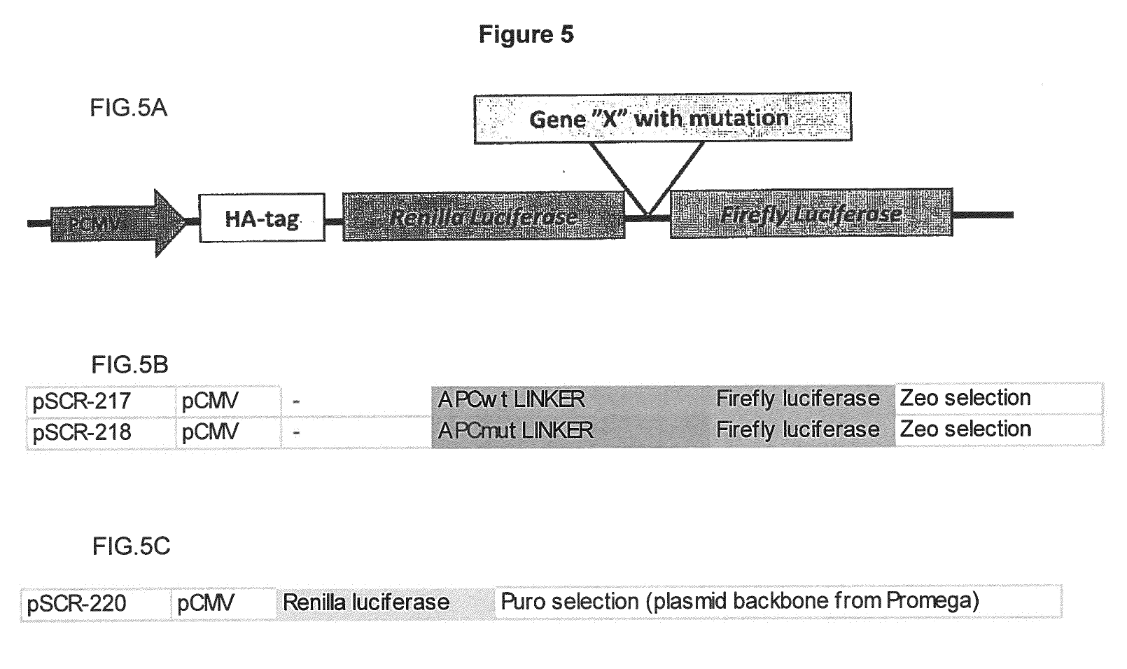

A similar reporter construct design also can be used to identify compounds that override premature stop codons in mammalian systems. In one embodiment, a dual construct can be made (see FIG. 5A) such that the fragment containing the premature stop codon is inserted in frame between a first reporter gene (e.g., Renilla luciferase) and a second reporter gene (e.g., Firefly luciferase). Both reporter genes are under the control of a strong constitutive promoter like PCMV. In addition, a fusion tag like HA can be added in frame to the first reporter gene to allow detection of the protein product using anti-HA directed antibodies. In this scenario, the Renilla luciferase reporter gene serves as an indicator for transfection efficiency and general expression level from construct and the Firefly luciferase reporter gene serves as an indicator for degree of translational read-through of premature stop codon.

Stable clones of mammalian HCT-116 cells containing integrated APC premature stop codon (C1450X) Firefly luciferase reporter constructs were generated and characterized. Such mammalian cell lines can serve as a secondary APC assay to screen extracts or compounds from hits identified from a screening campaign (for example, compound enabled yeast (CEY), i.e., a yeast strain transformed with yeast artificial chromosome; see Naesby et al., Microbial Cell Factories 8:45 pages 1-11 (2009); Klein et al., ACS Synthetic Biology 3:314-23 (2014)). Plasmids pSCR-217 and pSCR-218 were constructed with an APC (either WT or mutant) linker inserted in front of and in frame with a Firefly luciferase gene (optimized for mammalian cell expression). The APC reporter was expressed from a strong constitutive CMV promoter and harbors the ZeoR selection gene. See FIG. 5B for a diagram of plasmids. Linkers used were similar to those used to construct the yeast APC plasmids. In addition, plasmid pSCR-220 (see FIG. 5C) was constructed with a Renilla luciferase gene under PCMV control and the puroR gene for selection. Introducing this plasmid in the cells allowed normalization of the Firefly luciferase activity, which was regulated by readthrough of the APC linker, to the Renilla luciferase activity which remains constant in the presence or absence of suppressing drugs.

Transfection of HCT-116 cells was performed using GeneJuice® reagent and cells were handled as described in FIG. 6. Isolated clones were seeded into 96-well dishes in media selecting for both plasmids in 2 sets (each in triplicate) where G418 was added to one set and vehicle added to the other set. The assay for luciferase activity was performed using the Dual Luciferase® Assay Kit from Promega according to manufacturer's guidelines. Table 2 below summarizes the status of stable HCT-116 clones with APC Firefly luciferase reporter constructs. Cell lines were expanded and frozen in ampules stored in liquid nitrogen for future use.

TABLE 2
HCT-116 mammalian cell clones.

| | Construct | Construct | | No |
|---|---|---|---|---|
| Cell clone: | 1: | 2: | Frozen: | vials: |
| HCT-116 P_{CMV}-Rluc | pSCR-220 | — | 9.7.12 (P4) | 11 |
| clone 5 | | | | |
| HCT-116 P_{CMV}-Rluc | pSCR-220 | — | 9.7.12 (P4) | 11 |
| clone 4 | | | | |
| HCT-116 P_{CMV}-Rluc | pSCR-220 | pSCR-117 | 9.7.12 (P4) | 15 |
| P_{CMV}-APCwt-Fluc clone 1 | | | | |
| HCT-116 P_{CMV}-Rluc | pSCR-220 | — | 25.6.12 | 17 |
| clone 1 | | | | |
| HCT-116 P_{CMV}- | | pSCR-118 | Not yet | |
| APCmut-Fluc clone B5 | | | | |

Thus, HCT-116 derived APCmut Fluc reporter clones were developed that responded to translational read-through compounds in preliminary tests that can be used as a secondary assay to screen future hits.

### Example 4. Screening of Primary Yeast-Based SCR Assay Strain—Identification and Characterization of Potential CEY Hits

A pilot screen was conducted with the yeast strains harboring a functional and validated APC premature stop codon assay. For this purpose EFSC2686 (mat a) and EFSC2683 (mat alpha) were transformed using the spheroplast method (see “Spheroplast transformation” of Naesby et al., Microbial Cell Factories 8:45 pages 1-11 (2009)) with eYACs made from a super pathway mix (eYAC pSCR-sp 11-06-12) and eYACs made from type III polyketide pathway+decoration enzymes mix (pSCR-pk+dec 11-06-12) respectively. Type III polyketides were chosen as magrolides belong to the polyketide family. In addition, EFSC2683 and EFSC2683 were transformed with empty HIS3 and LEU2 plasmids to serve as a “non-eYAC” control. Transformation with eYACs yielded about 2000 and 4000 transformants in EFSC2683 and EFSC2686, respectively. Transformants were harvested and stored as frozen SCR haploid library aliquots. Percentage of clones containing both eYAC arms was calculated (see Table 3 below).

TABLE 3
Calculation of arm percentage for eYAC haploid libraries

| | pk + dec |
|---|---|
| SC-his (colonies) | 168 |
| SC-his-trp (colonies) | 106 |
| % with both arms | 63 |
| | sp |
| SC-leu (colonies) | 144 |
| SC-leu-trp (colonies) | 50 |
| % with both arms | 35 |

Haploid library populations were expanded in liquid SC-His+2 mM Met or SC-Leu+2 mM Met media and mating as conducted in Naesby et al., Microbial Cell Factories 8:45 pages 1-11 (2009), and Klein et al., ACS Synthetic Biology 3:314-23 (2014)). Strains with empty plasmids were included as control. Following diploid expansion overnight, 2×10E8 cells were plated on SCgal-HLU+/−2 mM Met to allow testing for methionine (and thus eYAC) dependency. Yeast screening plate protocol is shown below in Table 4.

TABLE 4
Yeast screening plate protocol.
Primary screening plates for Ura3-reporter activation (07.06.12)

| Flask 1 | Flask 2 |
|---|---|
| SCgal-His-Leu-Ura-Met-plader 1000 mL | |
| 900 mL H2O | 20 g gal |
| 10 g Succinic Acid | 100 mL H2O |
| 6 g NaOH | |
| 6.7 g YNB | |
| 1.31 g SC-His-Leu-Ura-Trp-Met drop-out-mix | |
| 85.6 mg Trp = 856 uL (100 mg/mL) | |
| 20 g Bacto Agar | |
| Autoclave Flask 1 + 2 and cool to ~65° | |
| Then mix the 2 bottles | |
| SCgal-His-Leu-Ura + 2 mM Met-plader 1000 mL | |
| 900 mL H2O | 20 g gal |
| 10 g Succinic Acid | 100 mL H2O |
| 6 g NaOH | |
| 6.7 g YNB w/o aa but with ammoniumsulphate | |
| 1.66 g SC-His-Leu-Ura | |
| 10 mL Met-stock 200 mM | |
| 15 g Agar | |
| Autoclave Flask 1 + 2 and cool to ~65° | |
| Then mix the 2 bottles | |

Drop-out mixes were prepared with SC medium from Formedium (see above). As an example, SCgal-HLUM medium stands for Sc medium with galactose and without H (his=histidine), L (leu=leucine), U (ura=uracil), M (met=methionine). As indicated, in some cases also tryptophan was deleted (−trp).

A total of 504 Ura3p producing colonies (an average of 136 colonies/plate) were present on the 4 large SCgal-HLUM screening plates from the sp×pk+dec screen. In the presence of methionine, 107 colonies grew on the plate, a small reduction in the number of Ura3 producing colonies. For the control strain without eYACs, an average of 91 colonies grew per SCgal-HLUM screening plate. Thus there appears to be a higher number of Ura3p producers when eYACs are present.

Putative CEY hits from the sp×pk+dec screen were picked from the large SCgal-HLUM screening plates onto master plates (SC-HL+2 mM Met). From here, cells were grown in liquid SCgal-HLM cultures in 96-well format and re-tested for methionine-dependent effect on URA3 reporter expression (on SCgal-HLU+/−2 mM Met plates). Nineteen CEYs displayed a methionine-dependent growth on plates lacking uracil suggesting that the eYACs were producing a compound that permits transcription of the PGAL1-APCmut-URA3 reporter. These 19 CEYs were selected for more detailed URA3 reporter analysis as well as analysis of the firefly luciferase reporter system. Freezer stocks of these CEYs were also prepared and stored. Analysis of firefly luciferase activity in the 19 CEYs grown in the presence or absence of 2 mM Met was performed as described in the examples below. Control 11a is the diploid assay strain containing empty HIS3 and LEU2 plasmids.

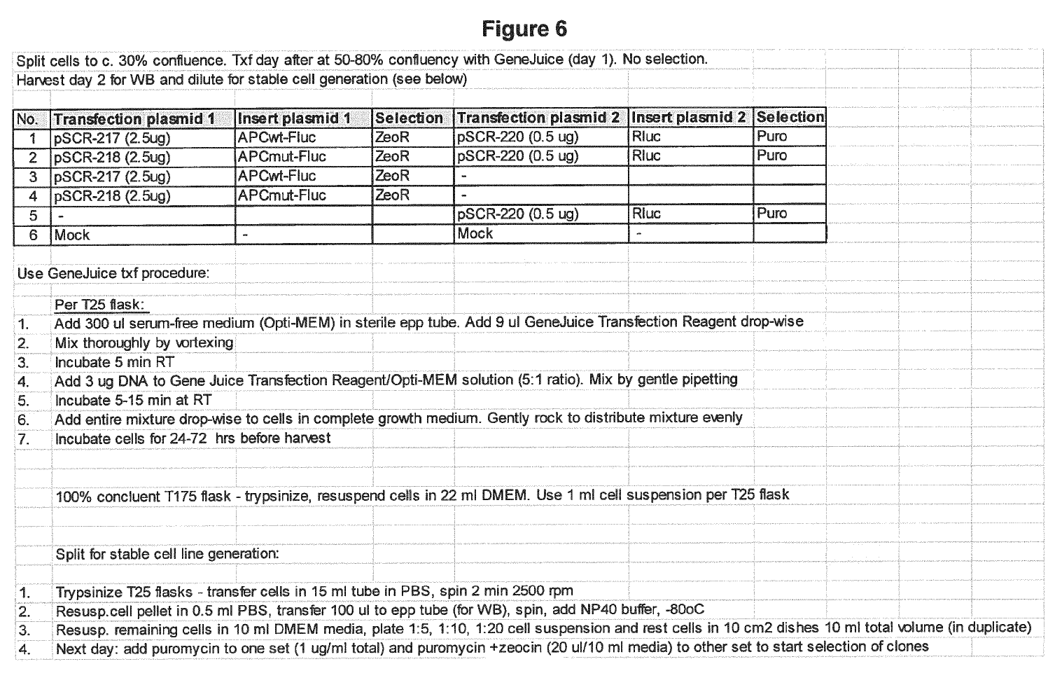

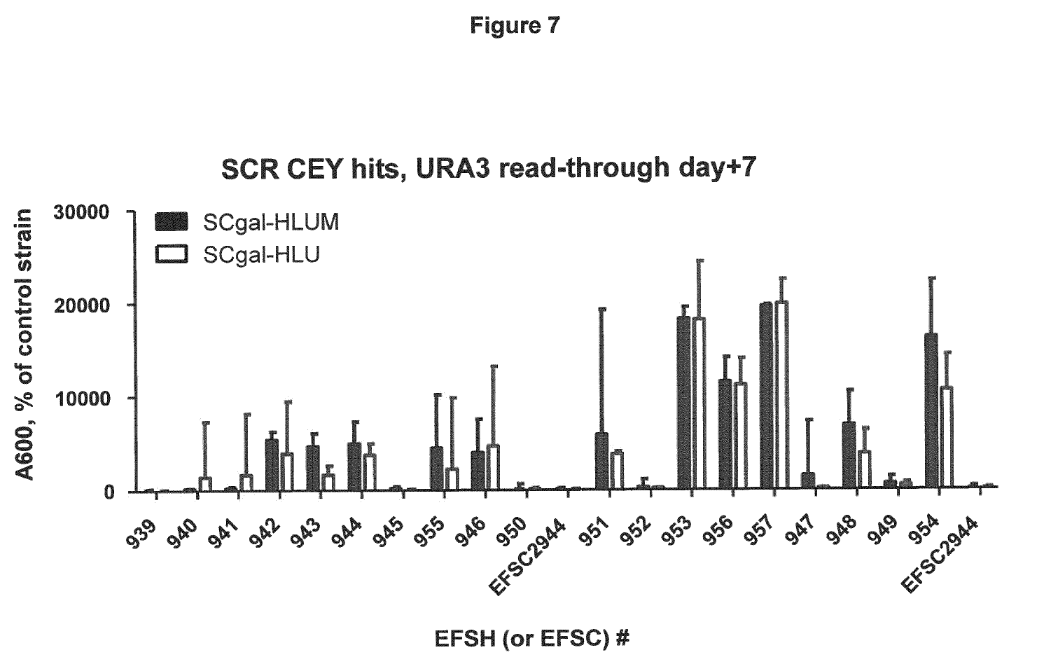

The CEYs were tested twice for eYAC effect on URA3 and firefly luciferase reporter genes (see also Table 5 with prioritized CEYs below). Results are shown in FIG. 7 (data tested in triplicate and analyzed by GraphPad Prism® software). Growth in SCgal-HLU and SCgal-HLUM is indicated as A600, % of control strain (EFSC2944). As evident, a high number of CEYs display growth in media lacking uracil, consistent with URA3 reporter transcription. A handful of CEYs show methionine-dependent growth in media lacking uracil consistent with an eYAC contribution. However, the observation that some CEYs show little methionine-dependence does not rule out a contribution from the eYAC, as it cannot be certain that 2 mM methionine over 7 days is sufficient to shut down eYAC gene expression. Therefore, all CEYs that are able to grow in the absence of uracil are potential CEY hits.

A majority of CEYs showed higher firefly luciferase activity compared to EFSC2944 control strain. Thus, from the pilot screen about 18 CEYs were isolated with the expected phenotype of a CEY hit. From these results, the following list was generated of CEYs prioritized for metabolite analysis based on the effects observed on both URA3 and firefly luciferase reporter in the 3 independent experiments conducted (1st, 2nd and 3rd test). Plus and minus signs indicate intensity of signal/growth (‘++++’ strongest to ‘+’ as weakest, and ‘-’ indicates no growth): ‘++++’ are the stronger performers showing the highest level of growth and luciferase activity, whereas ‘+++’, ‘++’ or ‘+’ are weaker performers showing less growth and luciferase activity, while ‘-’ indicates no significant difference from control strain. Only one CEY (EFSH952) failed the test as it showed no significant difference from control strain in all 3 experiments. The number in the last row “Rank” indicates the priority number with 1 being the strongest performing CEY hit and 19 the CEY with lowest priority.

TABLE 5
SCR CEY hit priority list.

| | | 1^{st} test | | 2^{nd} test | 3^{rd} test | |
|---|---|---|---|---|---|---|
| | | Fluc | URA3 | Fluc | URA3 | ‘Rank’ |
| CEY4904 | EFSC939 | − | − | ++ | − | 14 |
| CEY4905 | EFSC940 | − | − | ++ | − | 15 |
| CEY4906 | EFSC941 | − | − | ++ | − | 16 |
| CEY4907 | EFSC942 | ++ | +++ | ++++ | ++ | 4 |
| CEY4908 | EFSC943 | ++ | +++ | ++++ | ++ | 5 |
| CEY4909 | EFSC944 | ++ | +++ | + | ++ | 6 |
| CEY4910 | EFSC945 | − | − | ++ | − | 17 |
| CEY4911 | EFSC946 | ++ | +++ | − | + | 10 |
| CEY4912 | EFSC947 | − | +++ | − | − | 12 |
| CEY4913 | EFSC948 | ++ | ++++ | − | ++ | 9 |
| CEY4914 | EFSC949 | − | +++ | − | − | 13 |
| CEY4915 | EFSC950 | − | − | − | + | 18 |
| CEY4916 | EFSC951 | ++ | +++ | + | ++ | 8 |
| CEY4917 | EFSC952 | − | − | − | − | 19 |
| CEY4918 | EFSC953 | ++++ | ++++ | ++ | +++ | 2 |
| CEY4919 | EFSC954 | ++++ | ++++ | + | ++ | 7 |
| CEY4920 | EFSC955 | − | +++ | − | − | 11 |
| CEY4921 | EFSC956 | ++++ | ++++ | − | +++ | 3 |
| CEY4922 | EPSC957 | ++++ | ++++ | ++ | ++++ | 1 |
| CEY4923 | Control SCR | | | | | |

### Yeast Growing and Extraction Conditions

Yeast cultures were grown in synthetic complete (SC) media containing 2% glucose. Pre-cultures (25 ml) were cultivated in shake flasks at 30° C. for 24 hours and subsequently diluted to an OD600=0.1 in 0.5 L cultures with similar media supplemented. The 0.5 L culture was grown in a shake flask at 30° C. for 72 hours, eYAC “switched on” conditions, before yeast cells were harvested by centrifugation separating cell pellet from supernatant. In parallel, a culture of a control strain containing no YACs was also grown. Metabolites were extracted from the supernatant by means of liquid-liquid extraction (LLE) in EtOAc, 1:1 Vol. The aqueous layer was discarded. The organic solvent layer was kept and evaporated under vacuum. Dry crude yeast extracts were dissolved in DMSO at concentrations of 100 mg/ml.

### Clone Selection

Based on the primary yeast-based SCR assay screening ‘clone hits’ were chosen for large batch cultures (0.5 L), semi-preparative HPLC fractionation and activity screening of HPLC fractions in the luciferase based assay. To test for the presence of active YAC-dependent compounds, yeast crude extracts were fractionated by semi-preparative RP-HPLC in a time dependent fashion using 96 well collection plates collecting 176 isochronous fractions over the HPLC run of 27 minutes. The reconstituted supernatant was injected in a semi-preparative HPLC column (XBridge C18, 19×250 mm, 5 μm, Waters). Mobile phases used were: A: 0.1% TFA in Water and B: 0.1% TFA in Acetonitrile. The separation of the target compounds was achieved by a gradient from 1% B to 100% B in 27 minutes. The collected fractions were dried in a Genevac HT12 evaporation system. All fractions were tested in a secondary luciferase assay as described.

HPLC fractions were reconstituted in 20 μL DMSO, 2 μL was added to each well and the yeast were grown for 24 h at 30° C. in a water-jacketed incubator shaking at 400 rpm. Positive ([DMSO]=1% v/v with [paramomycin]=500 μM) or negative ([DMSO]=1% v/v) controls were added on each plate. After 24 hours of incubation, OD600 was measured. If OD600 was within 20% of each other: Added 50 μL passive lysis buffer (PLB) and incubated at room temperature with vigorous shaking for 30 minutes. 50 μL of Luciferase Assay Buffer (LARII) was added, and the luminescence was measured on a PerkinElmer Envision 2104, using the Promega kit, according to manufacturers protocol. If there was more than 20% variation of OD600, the same number of cells from each well was pelleted prior to passive lysis buffer addition, then the same protocol was followed. Fractions active in the secondary assay were analysed by HPLC/MS and their TICs were compared to corresponding fractions of the control yeast. Following de-replication, apparent active, YAC-dependent peaks were purified by semi-preparative HPLC.

### Identification of YAC-Dependent Metabolites

Aliquots of the crude metabolite extracts were diluted to 5 mg/ml and 5 uL (corresponding to 25 ug crude extract) were analysed by analytical RP-HPLC/MS. The LC-MS system was composed of a UPLC (Waters) coupled with a Time-Of-Flight Mass Spectrometer (MicroTOF II, Bruker) equipped with an Electrospray Ion Source (ESI). The UPLC column used was an Acquity BEH, C18, 2.1×100 mm, 1.7 μm. Mobile phases were A: 0.1% Formic Acid in water and B: 0.1% Formic acid in Acetonitrile. The gradient used for screening was ranged from 1% B to 50% B in 12 min, then to 100% B in 3 minutes and was held at 100% for 2 minutes. Flow rate was held at 0.4 ml/min. Mass spectra were internally calibrated using Na-formate as calibrant. The metabolomes of the yeasts active in the primary screen and the control strain were compared using MS-Xelerator software (Version 2.4, MsMetrix). TIC peaks were extracted in a retention time range between 2 and 14 min. An absolute intensity threshold of 10000 amu was applied on the first isotope. The TICs of the crude extracts of the active yeasts were dereplicated against the TIC of the control strain for detection of YAC dependent peaks.

### Purification of Compounds for MS and NMR Characterization

Four liter yeast culture batches were grown and extracted using the same conditions as those described for 0.5 L batch preparation. The reconstituted supernatant was injected in a semi-preparative HPLC column (XBridge C18, 19×250 mm, 5 μm, Waters). Mobile phases used were: A: 0.1% TFA in Water and B: 0.1% TFA in Acetonitrile. The separation of the target compounds was achieved by a gradient from 1% B to 100% B in 27 min. The collected fractions were dried in a Genevac HT12 evaporation system and a final quality control (QC) was done in the LC-MS using the LC conditions described above.

### NMR Analysis of Compounds

All NMR experiments were performed in DMSO-d₆ at 25 C using a Bruker Avance III 600 MHz NMR spectrometer equipped with a 1.7 mm cryogenic TCI probe. The structures were solved by means of one- and two-dimensional standard homo- and heteronuclear multipulse NMR experiments, namely ¹H,¹H-COSY, ¹H,¹H-ROESY, ¹H,¹³C-HSQC and ¹H,¹³C-HMBC experiments.

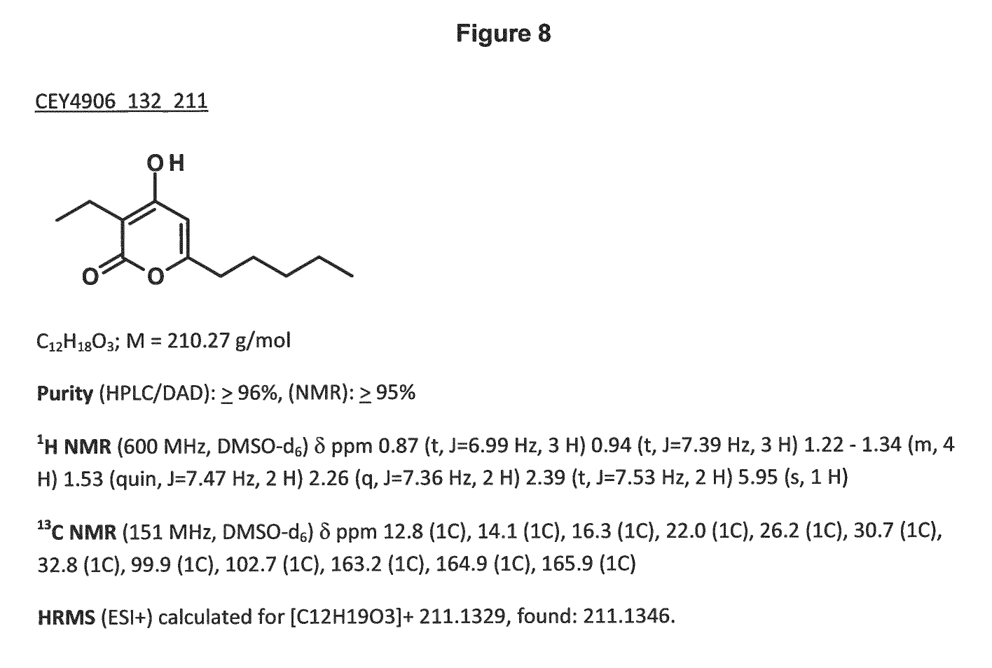

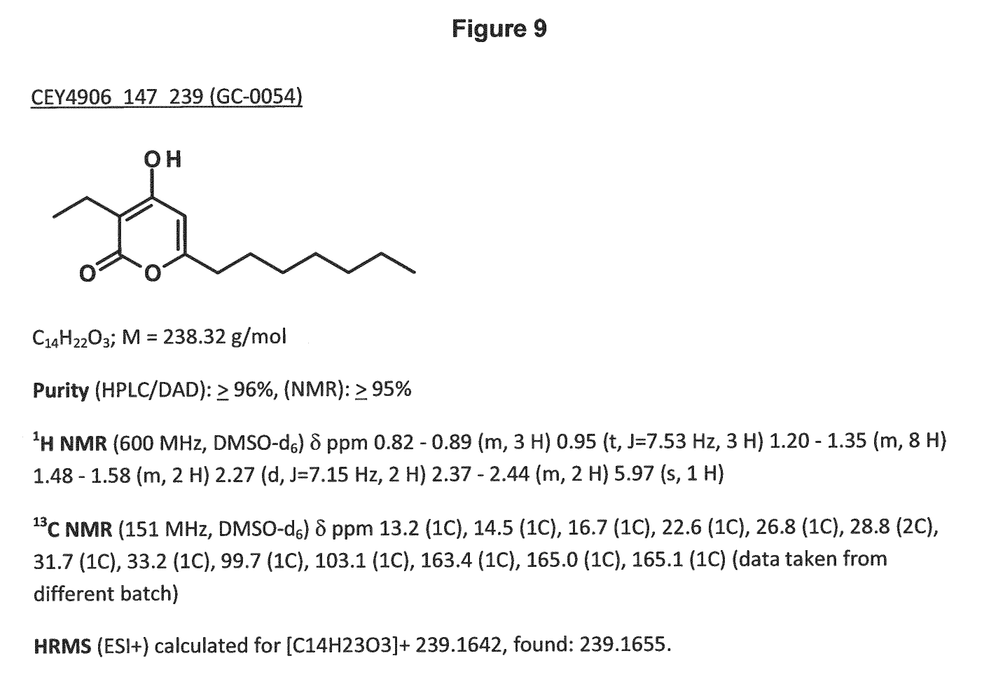

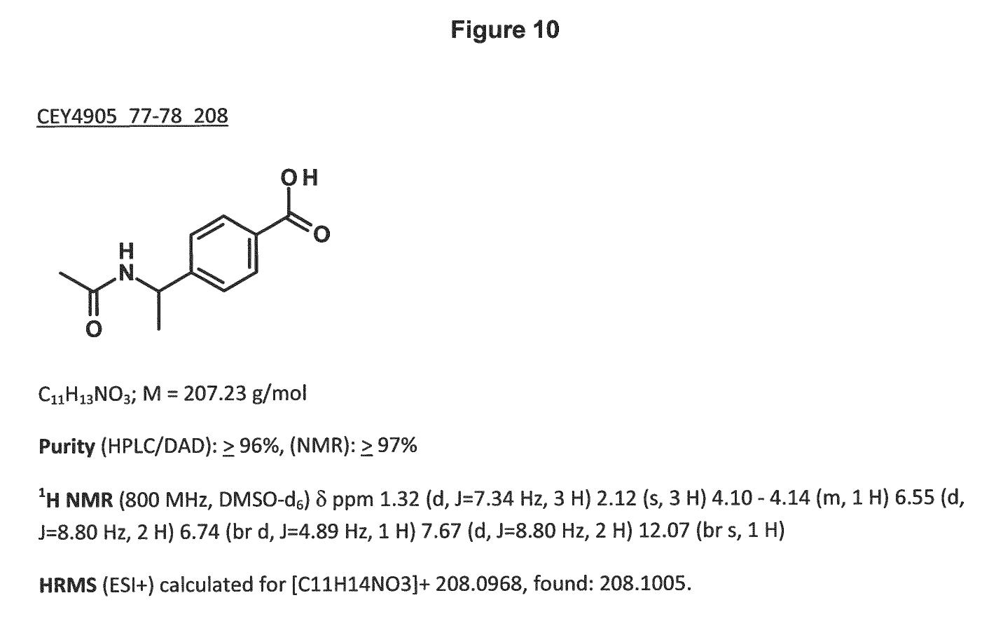

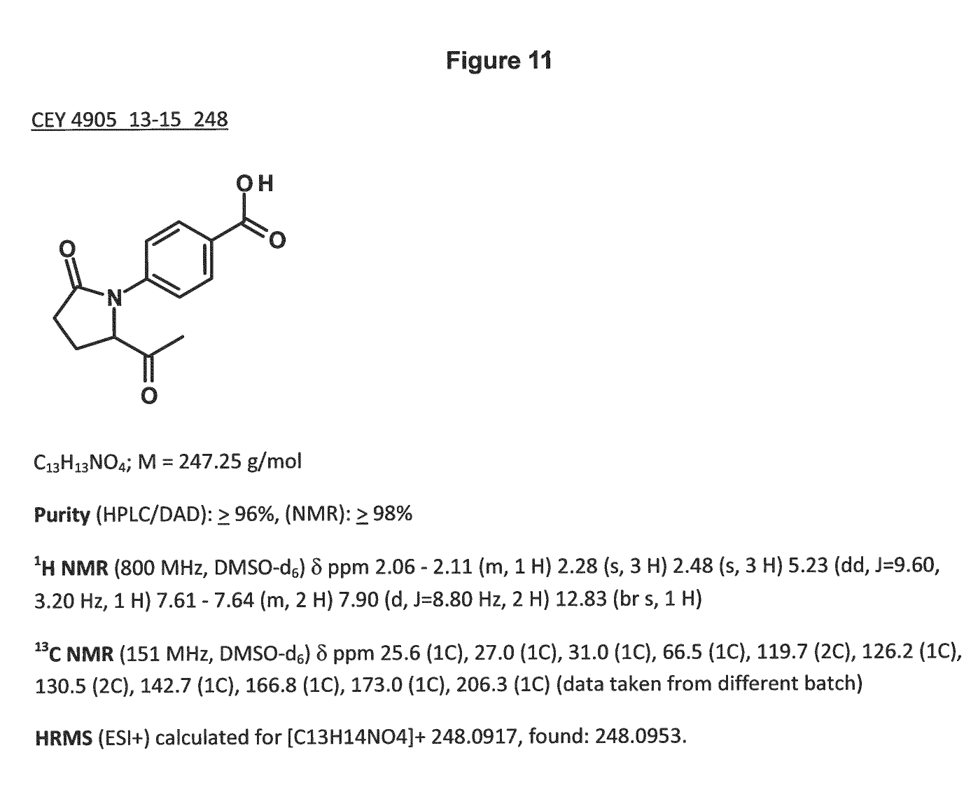

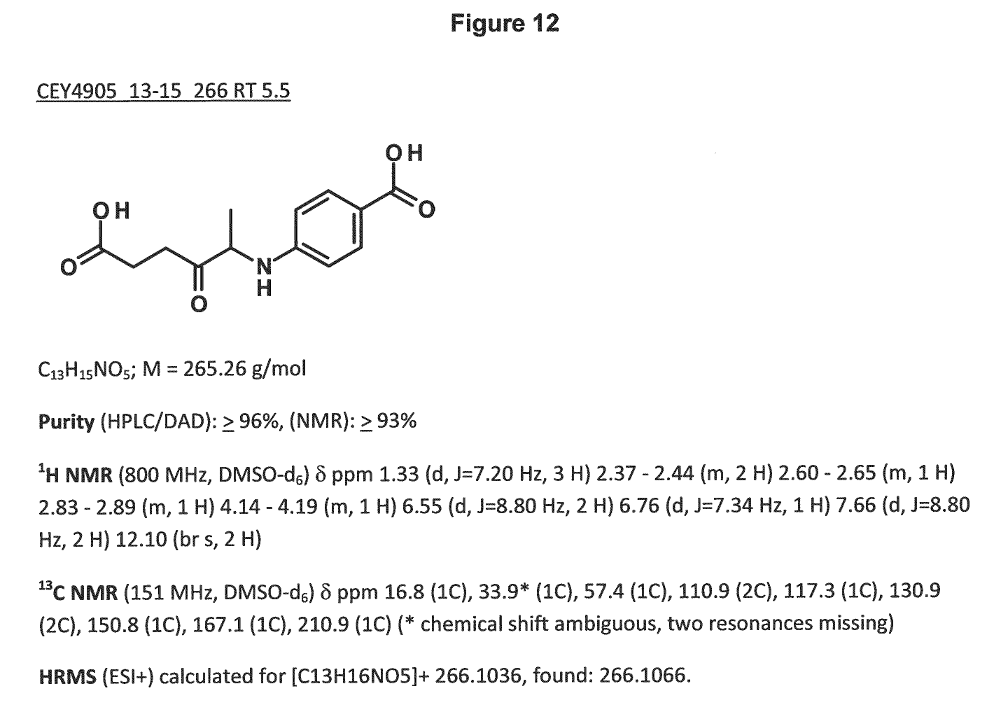

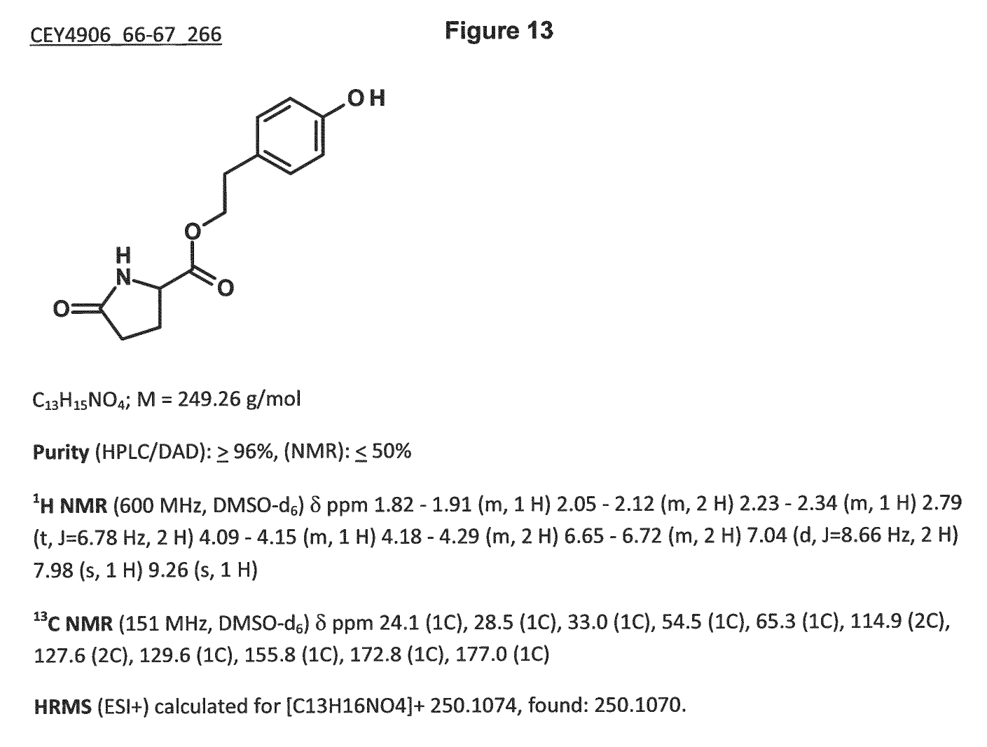

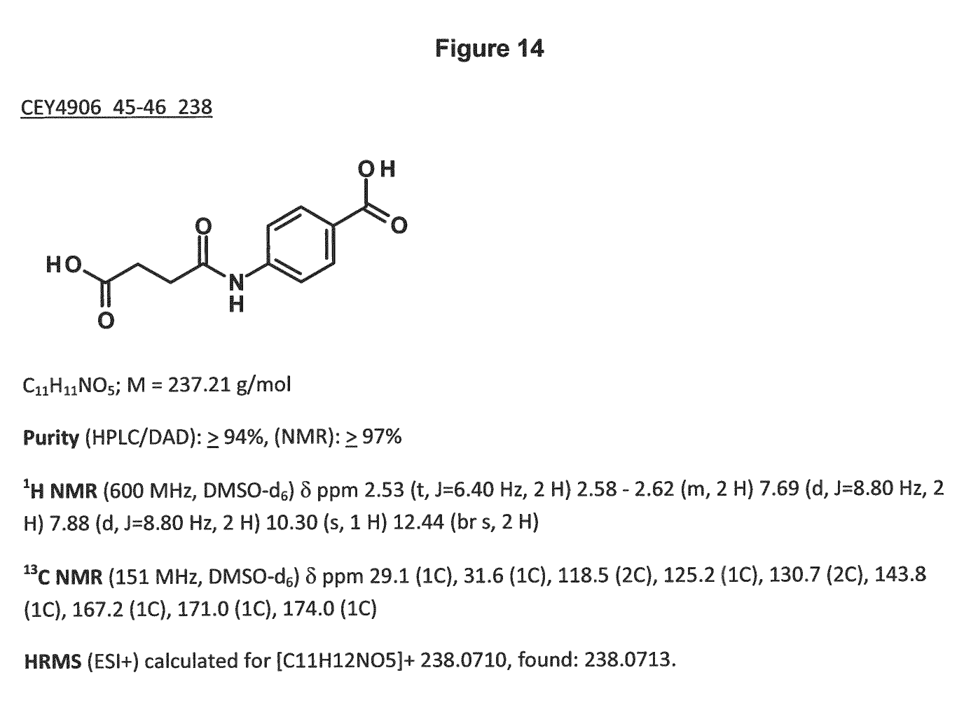

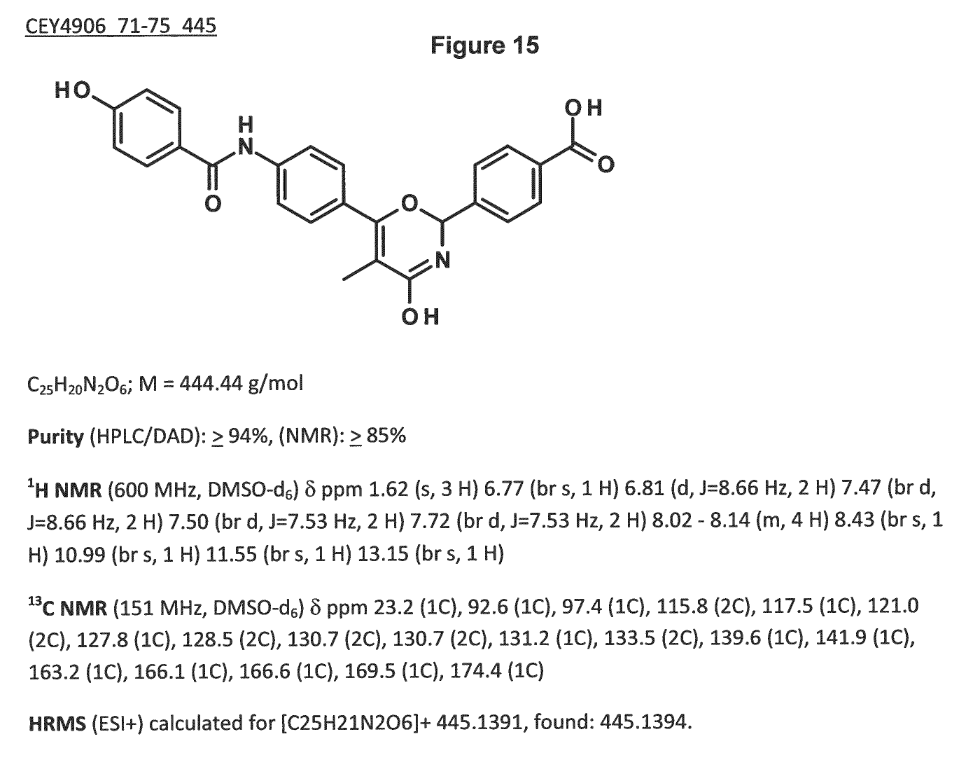

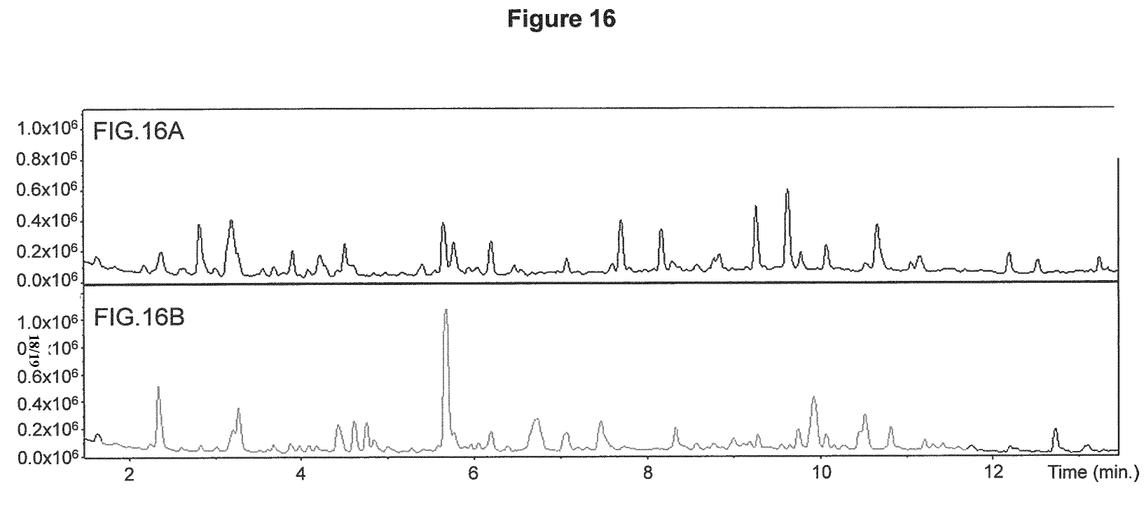

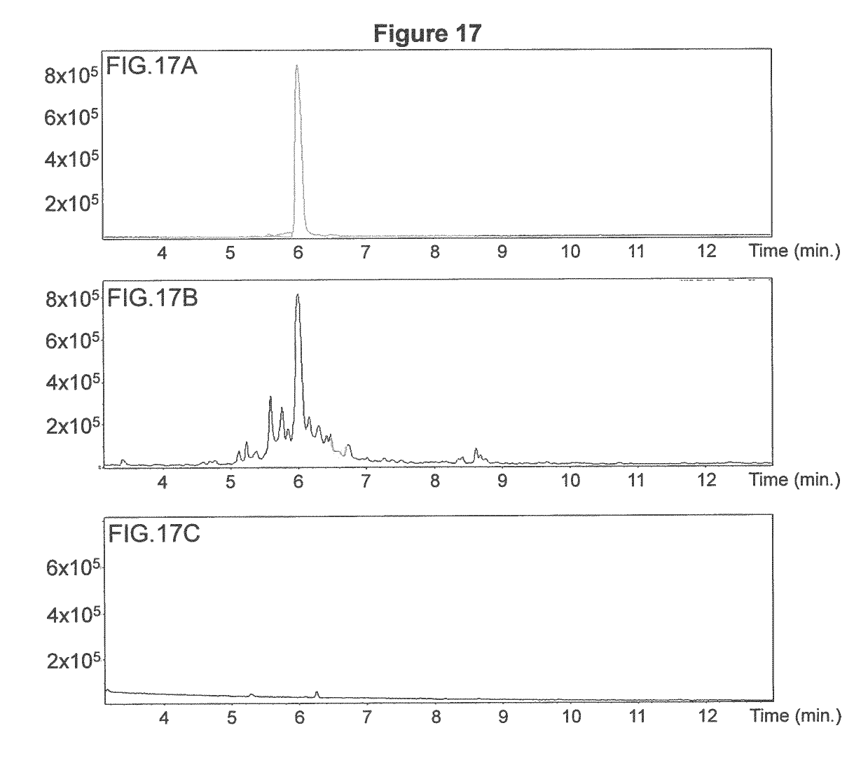

TABLE 6
Identified yeast-derived SCR compounds

| Compound | SMILES |
|---|---|
| CEY4906_132_211 | CCCCCc1cc(c(c(═O)o1)CC)O |
| (also see FIG. 8) | |
| CEY4906_147_239 | CCCCCCCc1cc(c(c(═O)o1)CC)O |
| (GC-0054) | |
| (also see FIG. 9) | |
| CEY4905_77-78_208 | CC(c1ccc(cc1)C(═O)O)NC(═O)C |
| (also see FIG. 10) | |
| CEY 4905_13-15_248 | CC(═O)C1CCC(═O)N1c2ccc(cc2)C(═O)O |
| (also see FIG. 11) | |
| CEY4905_13-15_266 | CC(C(═O)CCC(═O)O)Nc1ccc(cc1)C(═O)O |
| RT 5.5 | |
| (also see FIG. 12) | |
| CEY4906_66-67_266 | c1cc(ccc1CCOC(═O)C2CCC(═O)N2)O |
| (also see FIG. 13) | |
| CEY4906_45-46_238 | c1cc(ccc1C(═O)O)NC(═O)CCC(═O)O |
| (also see FIG. 14) | |
| CEY4906_71-75_445 | CC1=C(OC(N=C1O)c2ccc(cc2)C(═O)O)c3ccc(cc3)NC(═O)c4ccc(cc4)O |
| (also see FIG. 15) | |

Having described the invention in detail and by reference to specific embodiments thereof, it will be apparent that modifications and variations are possible without departing from the scope of the invention defined in the appended claims. More specifically, although some aspects of the present invention are identified herein as particularly advantageous, it is contemplated that the present invention is not necessarily limited to these particular aspects of the invention.

1. A method for identifying compounds that permit translational read-through of nonsense mutations, the method comprising:
(a) providing a recombinant microorganism, the microorganism comprising at least one nucleic acid construct, the construct comprising a regulatory region operably linked to a nucleic acid sequence containing a nonsense mutation, wherein the nucleic acid sequence containing the nonsense mutation is operably linked to a nucleic acid sequence encoding a first reporter gene;
(b) contacting the recombinant microorganism with a test compound; and
(c) determining if the microorganism produces the reporter.

2. The method of claim 1, wherein the reporter is a fluorescent, chemiluminescent bioluminescent, selectable auxotrophic or selectable antibiotic resistant molecule.

3. The method of claim 1, wherein the reporter is firefly luciferase, renilla luciferase, green fluorescent protein (GFP), HcRed, DsRed, cyan fluorescent protein (CFP), yellow fluorescent protein (YFP), blue fluorescent protein (BFP), glutathione-S-transferase (GST), horseradish peroxidase (HRP), chloramphenicol acetyltransferase (CAT) beta-galactosidase, beta-glucuronidase (GUS), orotidine-5′ phosphate decarboxylase, phosphoribosylaminoimidazole carboxylase, glutathione-S-transferase (GST), horseradish peroxidase (HRP), chloramphenicol acetyltransferase (CAT), beta-galactosidase or beta-glucuronidase (GUS).

4. The method of any one of claims 1 to 3, wherein the recombinant microorganism is prokaryotic or eukaryotic.

5. The method of any one of claims 1 to 4, wherein the recombinant microorganism is bacteria, yeast, fungi or mammalian cell lines.

6. The method of any one of claims 1 to 5, wherein the test compound is a polypeptide, peptide, antibody, peptidomimetic, peptoid, small inorganic molecule, small non-nucleic acid organic molecule, nucleic acid, carbohydrate or other agent.

7. The method of any one of claims 1 to 6, wherein the nonsense mutation of the nucleic acid construct is known to result in a genetic disorder in a subject.

8. The method of any one of claims 1 to 7, wherein the recombinant microorganism comprises a second nucleic acid construct comprising a promoter operably linked to a native gene containing a native stop codon that is operably linked to a second reporter gene, wherein the second reporter gene is a different reporter gene than the first reporter gene.

9. The method of claim 8, wherein the first reporter gene is firefly luciferase and the second reporter gene is renilla luciferase, or wherein the first reporter gene is green fluorescent protein and the second reporter gene is red fluorescent protein.

10. The method of claim 8 or 9, wherein a high ratio of the first reporter gene signal compared to the second reporter gene signal indicates a compound results in translation read-through of the nonsense mutation, but not the native stop codon, and is a positive hit.

11. A pharmaceutical composition comprising the compound identified by the method of any one of claims 1 to 10.

12. Use of the composition of claim 11 for treating a subject with a genetic disorder caused by nonsense mutation.

13. A compound that permits translational read-through of nonsense mutations, wherein the compound is identified using the method of any one of claims 1 to 10.

14. Use of the compound of claim 13 for treating a subject with a genetic disorder caused by nonsense mutation.

15. A nucleic acid construct comprising a regulatory region operably linked to a nucleic acid sequence containing a nonsense mutation, wherein the nucleic acid sequence containing the nonsense mutation is operably linked to a nucleic acid sequence encoding a first reporter gene.

16. The nucleic acid construct of claim 15, wherein the nonsense mutation of the nucleic acid construct is known to result in a genetic disorder.

17. A nucleic acid construct comprising a regulatory region operably linked to a native gene containing a native stop codon that is operably linked to a second reporter gene, wherein the second reporter gene is a different reporter gene than the first reporter gene.

18. The nucleic acid construct of claim 15 or claim 17, wherein the reporter is a fluorescent, chemiluminescent bioluminescent, selectable auxotrophic or selectable antibiotic resistant molecule.

19. The nucleic acid construct of any one of claims 15 to 18, wherein the reporter is firefly luciferase, renilla luciferase, green fluorescent protein (GFP), HcRed, DsRed, cyan fluorescent protein (CFP), yellow fluorescent protein (YFP), blue fluorescent protein (BFP), glutathione-S-transferase (GST), horseradish peroxidase (HRP), chloramphenicol acetyltransferase (CAT) beta-galactosidase, beta-glucuronidase (GUS), orotidine-5′ phosphate decarboxylase, phosphoribosylaminoimidazole carboxylase, glutathione-S-transferase (GST), horseradish peroxidase (HRP), chloramphenicol acetyltransferase (CAT), beta-galactosidase or beta-glucuronidase (GUS).

20. The nucleic acid construct of claim 17, wherein the first reporter gene is firefly luciferase and the second reporter gene is renilla luciferase, or wherein the first reporter gene is green fluorescent protein and the second reporter gene is red fluorescent protein.

21. A recombinant microorganism comprising at least one nucleic acid construct, the construct comprising a regulatory region operably linked to a nucleic acid sequence containing a nonsense mutation, wherein the nucleic acid sequence containing the nonsense mutation is operably linked to a nucleic acid sequence encoding a first reporter gene.

22. The recombinant microorganism of claim 21 further comprising a second nucleic acid construct, the second nucleic acid construct comprising a regulatory region operably linked to a native gene containing a native stop codon that is operably linked to a second reporter gene, wherein the second reporter gene is a different reporter gene than the first reporter gene.

23. The recombinant microorganism of claim 21 or 22, wherein the recombinant microorganism is prokaryotic or eukaryotic.

24. The recombinant microorganism of any one of claims 21 to 23, wherein the recombinant microorganism is bacteria, yeast, fungi or mammalian cell lines.

25. A method of treating a condition caused by a nonsense mutation in a subject, comprising administering to the subject a pharmaceutically effective amount of the composition of claim 11.

26. The use of an effective amount of the composition of claim 14 for use in a method of treating a condition caused by a nonsense mutation in a subject.

27. The method of claim 25 or the use of claim 26, wherein the condition is cancer, Autosomal dominant congenital cataract, Early onset retinal dystrophy, Usher syndrome, Beta-thalassemia, Hemophilia (A and B), Colorectal cancer, Xeroderma pigmentosa, Cystic fibrosis, Lysosomal storage disease, Methylmalonic aciduria (MMA), Nervous system disorders, Menkes syndrome, Ostegenesis imperfecta (OI), Duchenne muscular dystrophy, Primary ciliary dyskinesia (PCD), Ichthyosis vulgaris, Epidermolysis bullosa, RDEB (recessive dystrophic E. bullosa), Erythrokeratoderma, Pachyonychia congenita or Neurofibromatosis.


SEQUENCE LISTING (2 sequences)

SEQ ID NO: 1 - DNA, 50 bp, Artificial Sequence
  Synthetic oligonucleotide
ctagcaaaatgcaaacagctcaaaccaagcgagaagtacctaaaaataaa

SEQ ID NO: 2 - DNA, 50 bp, Artificial Sequence
  Synthetic oligonucleotide
ctagcaaaatgcaaacagctcaaaccaagtgagaagtacctaaaaataaa